(19) United States
(12) Patent Application Publication
(10) Pub. No.: US 2026/0165689 A1
(43) Pub. Date: Jun. 18, 2026

(54) METHOD OF ULTRASOUND MICROBUBBLE-ASSISTED LIQUID BIOPSY

(71) Applicants: Taipei City Hospital, Taipei City (TW); National Defense Medical University, TAIPEI (TW); National Taiwan University of Science and Technology, TAIPEI CITY (TW)

(72) Inventors: Chih-Hung WANG, New Taipei City (TW); Cheng-Ping SHIH, Taipei City (TW); Ai-Ho LIAO, TAIPEI CITY (TW)

(21) Appl. No.: 19/409,775

(22) Filed: Dec. 5, 2025

Related U.S. Application Data
(60) Provisional application No. 63/728,698, filed on Dec. 6, 2024.

(51) Int. Cl. A61B 10/00; C12Q 1/6876; G01N 1/40; G01N 33/68

(52) U.S. Cl. CPC A61B 10/0045; C12Q 1/6876; G01N 1/4077; G01N 33/6848; C12Q 2600/158; C12Q 2600/178; G01N 2001/4088; G01N 2570/00

(57) ABSTRACT

A method for ultrasound microbubble-assisted liquid biopsy is provided, including: providing an individual to be tested; providing a microbubble composition, in which the microbubble composition includes a medium and a plurality of microbubbles dispersed in the medium; placing the microbubble composition on a surface of the individual to be tested; placing an ultrasonic device in contact with the microbubble composition, and applying non-focused ultrasound to the microbubble composition to raise a temperature of the surface by no more than 3° C.; and placing an absorbent substrate on the surface of the individual to be tested and absorbing a liquid located inside the individual to be tested, thereby achieving a non-invasive liquid biopsy, wherein the absorbent substrate is free of protein component, and the absorption and subsequent release effect is greater than 90%.

### CROSS-REFERENCE TO RELATED APPLICATION

This application claims priority to U.S. Provisional Application Ser. No. 63/728,698, filed on Dec. 6, 2024, which is herein incorporated by reference in its entirety.

### BACKGROUND

### Field of Invention

The present invention relates to a method of liquid biopsy. More particularly, the present invention relates to a method of ultrasound microbubble-assisted liquid biopsy.

### Description of Related Art

Liquid biopsy is a technique that uses bodily fluids such as blood, saliva, urine, pleural and peritoneal fluid, and cerebrospinal fluid for molecular detection and analysis of biomarkers within the body. Currently, in clinical practice, protein biomarkers in the blood have been developed for application in cancer diagnosis and monitoring. For example, tumors release many substances such as DNA, RNA, proteins, or whole cells into the circulatory system, and these substances in the circulatory system can reflect the tumor's genotype, phenotype, and dynamic changes. However, most protein biomarkers still face problems of insufficient specificity and sensitivity, limiting their application in early detection and disease diagnosis.

Traditional biopsies require invasive procedures to obtain samples by cutting, although liquid biopsy uses bodily fluid samples, other bodily fluids besides saliva and urine still need to be sampled using an invasive needle aspiration method. Therefore, the related art really needs to be improved.

### SUMMARY

The present disclosure provides a method of ultrasound microbubble-assisted liquid biopsy, comprising: providing an individual to be tested; providing a microbubble composition, wherein the microbubble composition comprises a medium and a plurality of microbubbles dispersed in the medium; placing the microbubble composition on a surface of the individual to be tested; placing an ultrasonic device in contact with the microbubble composition, and applying non-focused ultrasound to the microbubble composition to raise a temperature of the surface by no more than 3° C.; and placing an absorbent substrate on the surface of the individual to be tested and absorbing a liquid located inside the individual to be tested, wherein the absorbent substrate is free of protein component.

In some embodiments, the method further comprises: placing the absorbent substrate in a solvent and centrifuging to obtain a supernatant and a precipitate, the precipitate comprising cell debris; and performing a substance analysis on the supernatant.

In some embodiments, the method further comprises: placing the absorbent substrate in a solvent and centrifuging to obtain a supernatant and a precipitate, the precipitate comprising cell debris; separating the supernatant by an exosome filtering to obtain a plurality of exosomes; and analyzing proteins, nucleotides, or a combination thereof of each one of the plurality of exosomes.

In some embodiments, the exosome filtering comprises size exclusion chromatography, ultracentrifugation, sucrose density gradient centrifugation, ultrafiltration, polymer precipitation, immuno-magnetic bead capture, or microfluidic chip method.

In some embodiments, the medium comprises water, gel, or a combination thereof.

In some embodiments, a material of the plurality of microbubbles comprises albumin, polymer, lipid or a combination thereof.

In some embodiments, particle sizes of the plurality of microbubbles are from 0.5 micrometers (μm) to 2.5 μm.

In some embodiments, the ultrasonic device generates from 1 W/cm² to 5 W/cm².

In some embodiments, concentrations of the plurality of microbubbles in the microbubble composition range from 1×10⁶ particles/mL to 2×10⁸ particles/mL.

In some embodiments, the surface of the individual to be tested comprises skin, palpebra, nasal mucosa, or oral mucosa.

In some embodiments, the absorbent substrate comprises Schirmer strip, low-lint lab wipes, cellulose filter paper, glass fiber filter paper, cleanroom wipes, or a combination thereof.

The present disclosure also provides a method of ultrasound microbubble-assisted liquid biopsy, comprising: providing a microbubble composition, wherein the microbubble composition comprises a medium and a plurality of microbubbles dispersed in the medium; providing a model, comprising: a connecting pipe configured to imitate an inner ear structure, the connecting pipe comprising a first end and a second end opposite to the first end, configured to imitate inner ear structure; a dialysis membrane disposed on the second end of the connecting pipe, configured to imitate round window membrane; and a fixing ring comprising a ring portion and an accommodating portion, wherein the ring portion sleeved on an outer wall of the second end having the dialysis membrane, configured to imitate middle ear cavity; filling a liquid into the connecting pipe; placing the microbubble composition in the accommodating portion and located on the dialysis membrane; placing an ultrasonic device in contact with the microbubble composition, and applying a non-focused ultrasound to the microbubble composition to raise a temperature of the dialysis membrane by no more than 3° C.; and placing an absorbent substrate on the dialysis membrane, and absorbing the liquid in the connecting pipe, wherein the absorbent substrate is free of protein component.

The present disclosure also provides a method of ultrasound microbubble-assisted liquid biopsy, comprising: providing an individual to be tested, and then trepanning from a skull behind an ear to the middle ear cavity to expose a round window membrane; providing a microbubble composition, wherein the microbubble composition comprises a medium and a plurality of microbubbles dispersed in the medium; placing the microbubble composition on a surface of the round window membrane of the individual to be tested, and the surface located in the middle ear cavity; placing an ultrasonic device in contact with the microbubble composition, and applying non-focused ultrasound to the microbubble composition to raise a temperature of the surface by no more than 3° C.; and placing an absorbent substrate on the surface of the round window membrane of the individual to be tested, the surface located in a middle ear cavity, and absorbing a lymph located in an inner ear of the individual to be tested, wherein the absorbent substrate is free of protein component.

In some embodiments, the method further comprises: placing the absorbent substrate in a solvent and centrifuging to obtain a supernatant and a precipitate, the precipitate comprising cell debris; and performing a substance analysis on the supernatant.

In some embodiments, the method further comprises: placing the absorbent substrate in a solvent and centrifuging to obtain a supernatant and a precipitate, the precipitate comprising cell debris; separating the supernatant by an exosome filtering to obtain a plurality of exosomes; and analyzing proteins, nucleotides, or a combination thereof of each one of the plurality of exosomes.

In some embodiments, the exosome filtering comprises size exclusion chromatography, ultracentrifugation, sucrose density gradient centrifugation, ultrafiltration, polymer precipitation, immuno-magnetic bead capture, or microfluidic chip method.

In some embodiments, a material of the plurality of microbubbles comprises albumin, polymer, lipid or a combination thereof.

In some embodiments, particle sizes of the plurality of microbubbles are from 0.5 μm to 2.5 μm.

In some embodiments, concentrations of the plurality of microbubbles in the microbubble composition range from 1×10⁶ particles/mL to 2×10⁸ particles/mL.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion. The disclosure can be more fully understood by reading the following detailed description of the embodiment, with reference made to the accompanying drawings as follows:

FIG. 1 illustrates a flowchart of a microbubble-assisted liquid biopsy method according to some embodiments of the present disclosure.

FIG. 2A illustrates a schematic view of an in vitro bodily fluid collection model according to some embodiments of the present disclosure.

FIG. 2B illustrates a schematic view of a portion of the in vitro bodily fluid collection model during ultrasound administration according to some embodiments of the present disclosure.

FIG. 2C illustrates a schematic view of a portion of the placement of Schirmer strip in the in vitro bodily fluid collection model according to some embodiments of the present disclosure.

FIG. 2D illustrates a bar graph of the results of collecting fluorescein isothiocyanate-dextran (FITC-dextran) of different molecular weights in the in vitro bodily fluid collection model using Schirmer strip according to some embodiments of the present disclosure.

FIG. 3A illustrates a flowchart of a microbubble-assisted cochlear perilymph biopsy method according to some embodiments of the present disclosure.

FIG. 3B illustrates a schematic view of lymph sampling according to some embodiments of the present disclosure.

FIG. 4A illustrates a Venn diagram of the perilymph proteinosome analysis of some embodiments of the present disclosure.

FIG. 4B illustrates a schematic view of the functional groups of the perilymph proteinosome analysis of some embodiments of the present disclosure, with the left column of boxes corresponding to the right column of text descriptions in sequence.

FIG. 4C illustrates a schematic view of the subcellular locations of the perilymph proteinosome analysis of some embodiments of the present disclosure, with the left column of boxes corresponding to the right column of text descriptions in sequence.

FIG. 5 illustrates the top 25 most expressed proteins in the perilymph proteinosome analysis of some embodiments of the present disclosure.

FIG. 6 illustrates the Western blot used to verify the presence of cochlin protein in some embodiments of the present disclosure.

FIG. 7 illustrates the perilymph exosome in some embodiments of the present disclosure.

FIG. 8A illustrates a Venn diagram of proteomic analysis of perilymph exosome according to some embodiments of the present disclosure.

FIG. 8B illustrates a schematic view of the functional groups of the perilymph exosome for proteomic analysis in some embodiments of the present disclosure, with the left column of boxes corresponding to the right column of text descriptions in sequence.

FIG. 8C illustrates a schematic view of the subcellular locations of the perilymph exosome for proteomic analysis in some embodiments of the present disclosure, with the left column of boxes corresponding to the right column of text descriptions in sequence.

FIG. 9A illustrates a schematic view of the microRNA (miRNA) analysis of the perilymph exosome in some embodiments of the present disclosure.

FIG. 9B illustrates a schematic view of the microRNA (miRNA) analysis of the perilymph exosome in some embodiments of the present disclosure, with the left column of boxes corresponding to the right column of text descriptions in sequence.

FIG. 10 illustrates the top 20 most expressed miRNAs in the perilymph exosome analysis of some embodiments of the present disclosure.

### DETAILED DESCRIPTION

The following disclosure provides detailed description of many different embodiments, or examples, for implementing different features of the provided subject matter. These are, of course, merely examples and are not intended to limit the invention but to illustrate it. In addition, various embodiments disclosed below may combine or substitute one embodiment with another, and may have additional embodiments in addition to those described below in a beneficial way without further description or explanation. In the following description, many specific details are set forth to provide a more thorough understanding of the present disclosure. It will be apparent, however, to those skilled in the art, that the present disclosure may be practiced without these specific details.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, or “includes” and/or “including” or “has” and/or “having” when used in this specification, specify the presence of stated features, regions, integers, steps, operations, elements, and/or components, but do not preclude the presence or addition of one or more other features, regions, integers, steps, operations, elements, components, and/or groups thereof.

Further, when a number or a range of numbers is described with “about,” “approximate,” and the like, the term is intended to encompass numbers that are within a reasonable range considering variations that inherently arise during manufacturing as understood by one of ordinary skill in the art. For example, the number or range of numbers encompasses a reasonable range including the number described, such as within +/−10% of the number described, based on known manufacturing tolerances associated with manufacturing a feature having a characteristic associated with the number. This repetition is for the purpose of simplicity and clarity and does not in itself dictate a relationship between the various embodiments and/or configurations discussed.

The present disclosure utilizes a topical application of microbubble components to a surface of an individual (e.g., skin, palpebra, nasal mucosa, oral mucosa, or round window membrane) followed by ultrasound to avoid the systemic side effects that may occur with intravenous microbubble injection. Furthermore, the present disclosure applies a non-focused ultrasound administration to the surface of the individual, resulting in low energy and a surface temperature increase of no more than 3° C. (i.e., less than 3° C.). Focused ultrasound, on the other hand, uses locally high energy with a surface temperature increase exceeding 3° C. (i.e., greater than or equal to 3° C.), which could burn tissues and organs. The present disclosure also utilizes a protein-free absorbent substrate with an absorption and release efficiency greater than 90%. This absorbent substrate is placed on the side of the round window membrane of the individual being tested that is located in the middle ear cavity, absorbing liquid. The absorbent substrate is protein-free and has an absorption and release efficiency greater than 90%, thereby absorbing any overflowing lymph liquid from inside of the individual to be tested to avoid affecting the test results.

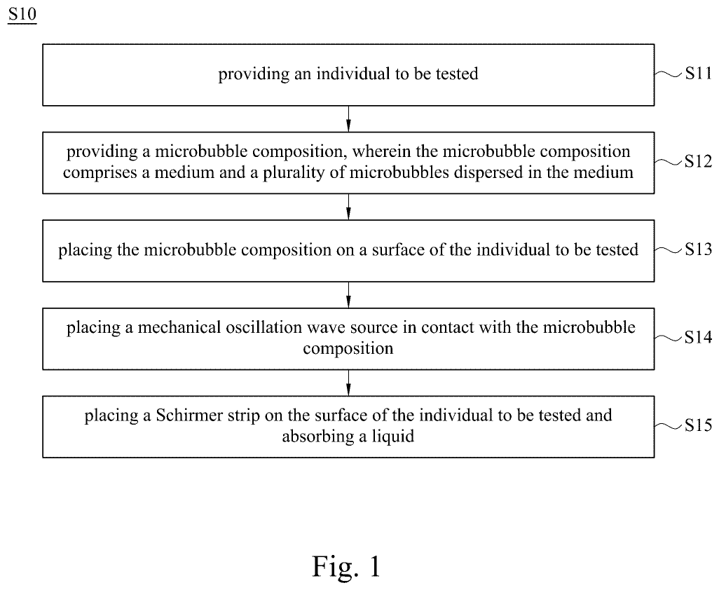

Some embodiments of the present disclosure provide a method S10 for microbubble-assisted liquid biopsy (FIG. 1), comprising: step S11, providing an individual to be tested; step S12, providing a microbubble composition, wherein the microbubble composition includes a medium and a plurality of microbubbles dispersed in the medium; step S13, placing the microbubble composition on the surface of the individual to be tested; step S14, placing a mechanical oscillation wave source in direct or indirect contact with the microbubble composition; and step S15, placing a test strip on the surface of the individual to be tested and absorbing liquid.

In some embodiments, a mechanical wave is generated by a mechanical oscillation wave source. The mechanical wave induces cavitation in the plurality of microbubbles in the microbubble composition, thereby increasing the surface transparency of the individual to be tested. In some embodiments, the mechanical wave induces cavitation in the plurality of microbubbles in the microbubble composition located on the surface of the individual to be tested.

In some embodiments, substance analysis includes, but is not limited to, protein analysis or nucleotide analysis, wherein nucleotide analysis includes DNA analysis or RNA analysis, wherein the RNA analysis includes miRNA analysis.

In some embodiments, the medium comprises water, gel, or a combination thereof. In some embodiments, the gel comprises, but is not limited to, gelatin, cellulose gel (e.g., carboxymethyl cellulose (CMC) or hydroxypropyl methylcellulose (HPMC) etc.), polysaccharide gel (e.g., alginate, hyaluronic acid (HA)), synthetic polymer gel (e.g., polyethylene glycol (PEG) hydrogel, polyvinyl alcohol (PVA) gel), or a combination thereof.

In some embodiments, the material of the plurality of microbubbles comprises alcohol, polymer, lipid, or a combination thereof. In some embodiments, lipid includes, but is not limited to, phospholipids (e.g., liposomes, DSPC (1,2-distearoyl-sn-glycero-3-phosphocholine), DPPC (1,2-dipalmitoyl-sn-glycero-3-phosphocholine)), PEGylated lipids, fatty acids (e.g., stearic acid or palmitic acid), cholesterol and its derivatives, or a combination thereof.

As used herein, “absorbent substrate” refers to a substrate that does not contain a protein component, or a substrate that contains a protein component but does not release its protein.

In some embodiments, the selection of the absorbent substrate is mainly based on its strong liquid adsorption capacity and the protein release rate after centrifugation being greater than 90%, which meets the selection criteria for absorbent substrate. That is, the protein release rate of the absorbent substrate is greater than 90%, for example 90%, 91%, 92%, 93%, 94%, 95%, 96%, 97%, 98%, 99%, or any value between any two of these values, and the absorbent substrate itself does not contain protein component. Substrates containing protein component, such as cotton, gauze, etc., are not the substrates intended to be used in the present disclosure. The absorbent substrate includes, but is not limited to, Schirmer strip, low-lint lab wipes, cellulose filter paper, glass fiber filter paper, cleanroom wipes, or a combination thereof. In some embodiments, the low-lint lab wipes include, but is not limited to, Kimwipes (Kimberly-Clark), KIMTECH Science Precision Wipes, or KIMTECH Sterile Wipes. In some embodiments, the cellulose filter paper includes, but is not limited to, Whatman Grade 1 (GE/Cytiva) or Whatman Grade 3. In some embodiments, the cellulose filter paper includes, but is not limited to, Whatman glass fiber filter paper (GF/F or GF/A). In some embodiments, the cleanroom wipes include, but are not limited to, Texwipe TX609 wipes, Texwipe TX3212 wipes, Bemcot M-1 cotton wipes (Asahi Kasei Corporation, Japan), or Bemcot M-1 cotton wipes (Asahi Kasei Corporation, Japan).

As used herein, the term “cavitation” indicates that ultrasonic waves of a certain energy and audio are applied to the microbubbles, which will induce the cavitation. Cavitation can be divided in to two types, one is called stable cavitation, also called non-inertial cavitation: when the microbubbles are repeatedly compression and rarefaction under the action of the low acoustic energy of the ultrasonic wave, the liquid around the microbubbles will flow, so the drug delivery can be promoted. The other is called inertial cavitation: when the microbubbles are extremely compression and rarefaction under the action of the strong acoustic energy of the ultrasound, so that the pulse wave and the liquid jet are generated by the final collapse of the microbubbles, and these forces can enhance the drug absorption at the target site.

The microbubble ultrasound contrast agent of some embodiments of the present disclosure may be in the form of an aqueous solution or a gel form. The material of the microbubbles can be roughly divided into three categories: albumin microbubbles, lipid microbubbles or polymer microbubbles. The microbubbles contained in the microbubble ultrasound contrast agent have stable shells and may be used to enhance the scattering signals of reflected ultrasound. Under various ultrasound energy intensities, using the microbubble ultrasound contrast agent can increase the permeability of the body surface.

In some embodiments, the lipid microbubbles comprise lipid microbubbles made of sulfur hexafluoride. In some embodiments, the lipid microbubbles comprise, but are not limited to, SonoVue® (Bracco, Milano, Italy). Before use, 25 mg of sonoVue cryocrystalline powder was mixed with 5 mL of saline to prepare a suspension according to the manufacturer's instructions. The prepared microbubbles contain 1×10⁸ microbubbles/mL to 5×10⁸ microbubbles/mL with an average diameter of 2.5 μm.

In some embodiments, the microbubble composition as used herein is also called the microbubble ultrasound contrast agent.

In some embodiments, the concentration of microbubbles in the microbubble composition is from 1×10⁶ to 2×10⁸ particles/mL. In one embodiment, the concentration is from 2×10⁶ to 2×10⁸ particles/mL. In one embodiment, the concentration is from 2×10⁷ to 2×10⁸ particles/mL. In one embodiment, the concentration is 1×10⁶, 2×10⁶, 3×10⁶, 4×10⁶, 5×10⁶, 6×10⁶, 7×10⁶, 8×10⁶, 9×10⁶, 1×10⁷, 1.5×10⁷, 2×10⁷, 3×10⁷, 4×10⁷, 5×10⁷, 5.5×10⁷, 6×10⁷, 7×10⁷, 8×10⁷, 9×10⁷, 1×10⁸, 2×10⁸ particles/mL, or any value between any two of these values. In which the concentration ranging from 1.5×10⁷ microbubbles/mL to 5.6×10⁷ microbubbles/mL was obtained based on the manufacturer's information and diluted tenfold. Experiments have shown that the effects of application within this range are similar.

In some embodiments, the microbubble composition of the present disclosure comprises a medium and a plurality of microbubbles dispersed in the medium.

In some embodiments, the microbubble composition of the present disclosure is administrated to the specific cavity, such as middle ear cavity. In one embodiment, the microbubble composition is applied to the middle ear cavity, and ultrasound is applied and in contact with the microbubble composition to enhance the permeability of the round window membrane and absorb the inner ear perilymph via the absorbent substrate. In some embodiments, the microbubble composition is applied to the middle ear cavity, and ultrasound is applied and in direct contact with the microbubble composition. In some embodiments, the microbubble composition is applied into the middle ear cavity, and ultrasound is applied and in indirect contact with the microbubble composition, for example, by placing an ultrasound probe in the ear canal (which is filled with a medium, such as water, gel, etc.) or placing the ultrasound probe on the skull behind the ear to apply ultrasound, with the ultrasound energy being transferred to the microbubble composition in the middle ear cavity, thus indirect ultrasound application is achieved.

As used herein, “perilymph” exists between the bony and membranous labyrinths of the inner ear and is rich in sodium ions; while “endolymph” exists in the cavities of the membranous labyrinth (such as the cochlea and semicircular canals) and is rich in potassium ions. The two work together to maintain the sensory functions of hearing and balance by generating a potential difference through ion differences.

In some embodiments, the time for which the absorbent substrate is placed on the surface after applying ultrasound is not particularly limited, as long as the time required to separate and analyze the substance from the absorbent substrate is sufficient. Absorption time includes, but is not limited to, 30 seconds to 60 minutes, for example, 30 seconds, 1 minute, 5 minutes, 10 minutes, 15 minutes, 20 minutes, 25 minutes, 30 minutes, 40 minutes, 50 minutes, 60 minutes, or any value between any two of these values.

Therefore, some embodiments of the present disclosure provide methods for delivering ultrasound waves via an ultrasound probe through transcanal or transcranial administration. Clinical applications and trials should, as far as possible, adopt non-invasive treatment methods. Therefore, the present disclosure provides a technique for delivering ultrasound waves by trepanning the skull behind the ear to the middle ear cavity to expose the round window membrane, thereby achieving a low-invasive method for inner ear lymph node biopsy.

In some embodiments, the ultrasound probe includes a transducer.

In some embodiments, the choice of ultrasonic energy is based on inducing the cavitation. Therefore, the choices of ultrasonic energy in both animal experiment and clinical experiment are adjusted based on the principle of inducing the cavitation. In one embodiment, ultrasonic energy includes, but is not limited to 0.1 W/cm² to 10 W/cm². In one embodiment, ultrasonic energy includes 0.5 W/cm²−5 W/cm², 1 W/cm²−5 W/cm², 1 W/cm²−4 W/cm², or 1 W/cm²−3 W/cm². In one embodiment, ultrasonic energy includes 0.1 W/cm², 0.2 W/cm², 0.3 W/cm², 0.4 W/cm², 0.5 W/cm², 0.6 W/cm², 0.7 W/cm², 0.8 W/cm², 0.9 W/cm², 1 W/cm², 1.5 W/cm², 2 W/cm², 2.5 W/cm², 3 W/cm², 3.5 W/cm², 4 W/cm², 4.5 W/cm², 5 W/cm², 5.5 W/cm², 6 W/cm², 6.5 W/cm², 7 W/cm², 7.5 W/cm², 8 W/cm², 8.5 W/cm², 9 W/cm², 9.5 W/cm², or 10 W/cm², or any value between any two of these values.

In some embodiments, the mechanical oscillation wave source includes, but is not limited to, an ultrasonic device, a laser device, or a combination thereof. In some examples, the ultrasonic device is a non-focused ultrasound (ST2000V, NepaGene, Japan). The ultrasonic probe is 6 mm in diameter, and the instrument waveform is a square wave with a duty cycle of 50%.

In some embodiments, surgical anesthesia is performed by inhalational anesthesia (Isoflurane); anesthesia for animal hearing function testing is performed by intramuscular injection of Zoltil combined with the muscle relaxant Rompun (xylazine), 10 mg/kg (Bayer, Shawnee Mission, KS, USA).

In some embodiments, the method for extracting proteins from perilymph involves cutting Schirmer strips into small fragments and placing them in 100-200 μL of phosphate-buffered saline (PBS). The mixture is then shaken at 8° C. for 30 minutes, centrifuged at 855×g (rpm) for 10 minutes and then at 10000×g for 20 minutes to remove cell debris and impurities. The supernatant is then collected and stored at −80° C. for subsequent analysis.

In some embodiments, the method for purifying exosomes from perilymph involves cutting Schirmer strips into small fragments and placing them in 100-200 μL of PBS. The mixture is then shaken at 8° C. for 30 minutes, centrifuged at 855×g (rpm) for 10 minutes and then at 10000×g (rpm) for 20 minutes to remove cell debris and impurities. A supernatant is then purified by passing it through a 35 nm qEV single column (IZON Science, New Zealand). PBS is used as the elution buffer, and fractions with higher exosome content are collected. These fractions are then concentrated using an Amicon® Ultra-4 centrifugal concentrator (Merck Millipore, USA). The concentrated exosomes are stored at −80° C. for subsequent experimental analysis.

In some embodiments, exosome RNA extraction and sequencing are performed using a qEV RNA Extraction Kit (IZON Science, New Zealand) for miRNA extraction, and the concentration is measured using a NanoDrop spectrophotometer. miRNA sequencing and library construction are performed using the QIAseq miRNA Library Kit on an Illumina® NovaSeq X Plus instrument, and performed normalization with cDNA of miRNA. Sequencing specifications: 75SE, 20M Raw Reads.

In some embodiments, the method for analyzing exosome number and particle size is to filter the purified exosome using a 0.22 μm filter, take 50 μL, and use a Litesizer™ 700 Dynamic Light Scattering (DLS; Anton Paar, Austria) instrument to measure the particle size distribution and concentration of the exosome, and then use a Multi-Angle Particle Sizing (MAPS) system for analysis and evaluation.

In some embodiments, protein quantification is performed by using the BCA protein assay (Thermo Scientific, USA) to separate bodily fluids (e.g., perilymph) and exosomes. The standard curve is 20-2000 μg/mL. After mixing the sample with the BCA reagent, the mixture is reacted at 37° C. for 30 minutes, and the absorbance (OD562) is measured at a wavelength of 562 nm. The protein concentration can be obtained from the standard curve.

In some embodiments, the method of enriching low-abundance proteins with magnetic nanoparticles involves using a low-abundance protein enrichment and pretreatment kit to enrich low-abundance proteins in perilymph samples. According to the manufacturer's instructions, 10 μL of magnetic nanoparticle suspension is taken, and the supernatant is removed after magnetic separation. The magnetic beads are resuspended after multiple washes, and 50 μL of separated perilymph is added. The mixture is then placed in a shaker at 37° C. for 30 minutes. The supernatant is then removed again by magnetic separation, and 150 μL of washing solution is added and the mixture is shaken for 5 minutes. This washing step is repeated three times. Next, reductive alkylation and enzymatic hydrolysis are performed to convert the protein into peptides, followed by C18 desalting. Finally, the total peptide concentration is determined using nanotitration.

In some embodiments, the data-independent acquisition (DIA) mode of mass spectrometry analysis involves proteomics data analysis being performed by Shanghai OE Biotechnology Co., Ltd. (Shanghai, China). A TimsTOF Pro2 mass spectrometer (Bruker) and a NanoElute (Bruker) system are used for shotgun proteomics and Data independent acquisition (DIA) experiments. Samples are loaded and separated on a C18 column (15 cm×75 μm ID, 1.6 μm C18) on the NanoElute (Bruker) system. The flow rate is set to 400 nL/min, and the linear gradient is 30 minutes. The linear gradient is set as follows: 0 -20 minutes, 5-22% B; 20-24 minutes, 22-37% B; 24-27 minutes, 37-80% B; 27-30 minutes, 80% B. For DIA, data acquisition is performed for 34 DIA windows. The collision energy is linearly adjusted according to the mobility, from 59 eV ramp at 1/K0=1.6 Vs cm⁻² to 20 eV at 1/K0=0.6 Vs cm⁻². The MS/MS spectral range is from 100 to 1700 m/z.

In some embodiments, data search and statistical analysis are performed using Spectronaut Pulsar 18.4 (Biognosys, Switzerland) with preset settings for searching and building a spectral library. These settings include using Trypsin/P as the enzyme, allowing a maximum of two missed cleavage sites, setting oxidation of methionine as a variable modification, cysteine carbamidomethylation (carbamidomethyl) as a fixed modification, and setting the FDR for PSM, peptide, and protein recognition to 1%. DIA data are analyzed using Spectronaut software with the aforementioned constructed spectral library. The main software parameters are set as follows: both the Precursor Qvalue cutoff and Protein Qvalue cutoff are set to 0.01, and MS2 is selected as the MS level (Quantity MS-Level) for quantification. The experimental groups are divided into a control group, a 3W-perilymph group, and a 3W-Schirmer strip group. The screening criteria for differentially expressed proteins (DEPs) were a fold change value greater than 1.2 or less than 0.83, and a p-value less than 0.05. Next, we identified the number of upregulated and downregulated proteins between each pair of groups. All identified proteins were annotated with GO (http://www.blast2go.com/b2ghome; http://geneontology.org/) and KEGG pathway (http://www.genome.jp/kegg/). Differentially expressed proteins were further enriched using GO and KEGG. Protein-protein interaction analysis was performed using the STRING database (https://string-db.org/).

In some embodiments, the auditory brainstem response (ABR) test was performed on guinea pigs before the experiment (Day 0), one week after surgery, and two weeks after surgery, by monitoring hearing thresholds. Animals were anesthetized by intramuscular injection of Zoltil combined with the muscle relaxant Rompun (xylazine), 10 mg/kg (Bayer, Shawnee Mission, KS, USA), and were kept warm using an electric heating pad during the experiment. After anesthesia, a needle electrode was inserted subcutaneously into the test ear, with the subcutaneous electrode of the other ear serving as the ground electrode, and the subcutaneous electrode on the vertex serving as the reference electrode. The stimulation method used a tone burst, with a stimulation duration of 10 milliseconds (ms). The 4k, 8k, 16k, and 32k frequency bands were measured, and thresholds were determined by decreasing the volume by 5 dB (SPL). The Tucker-Davis Technology system, System III was used.

In some embodiments, common exosome separation and purification methods are mainly classified into the following categories: size-exclusion chromatography, ultracentrifugation, sucrose density gradient centrifugation, ultrafiltration, polymer precipitation, immunoaffinity magnetic capture, microfluidics, etc.

In some embodiments, qEV single column (IZON Science, New Zealand) is a type of size exclusion chromatography. The column is packed with a porous polymer (typically a polysaccharide/agarose resin) as the stationary phase. When a sample (e.g., a biological liquid containing EVs) is added to the column, larger particles (such as EVs) cannot enter the resin micropores and therefore flow through quickly and elute first (in the “void volume”); conversely, smaller molecules (e.g., soluble proteins) can enter the pores and therefore have a longer retention time, eluting later.

A number of examples are provided herein to elaborate the method of microbubble-assisted liquid biopsy of the instant disclosure. However, the examples are for demonstration purpose alone, and the instant disclosure is not limited thereto.

### EXAMPLE

Although a series of operations or steps are used below to describe the method disclosed herein, an order of these operations or steps should not be construed as a limitation to the present disclosure. For example, some operations or steps may be performed in a different order and/or other steps may be performed at the same time. In addition, all shown operations, steps and/or features are not required to be executed to implement an embodiment of the present disclosure. In addition, each operation or step described herein may include a plurality of sub-steps or actions.

For the sake of clarity, features and elements that are well known in the art and are not necessary for an understanding of the principles described have been omitted.

### Example 1 in Vitro Bodily Fluid Collection Model

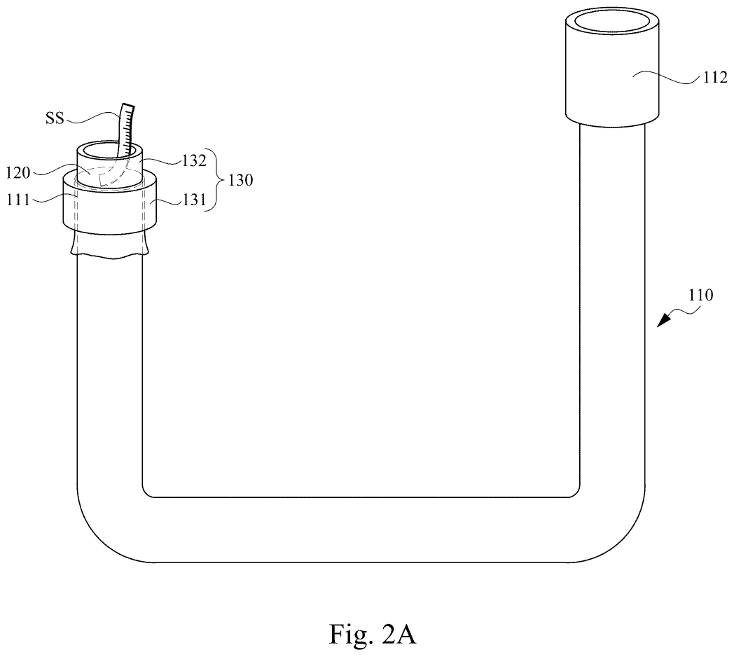

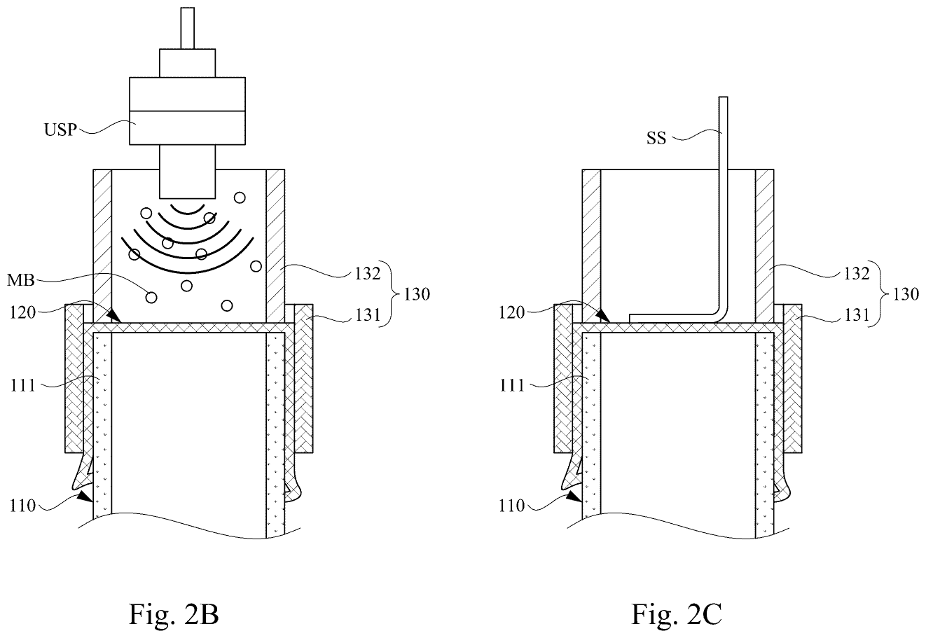

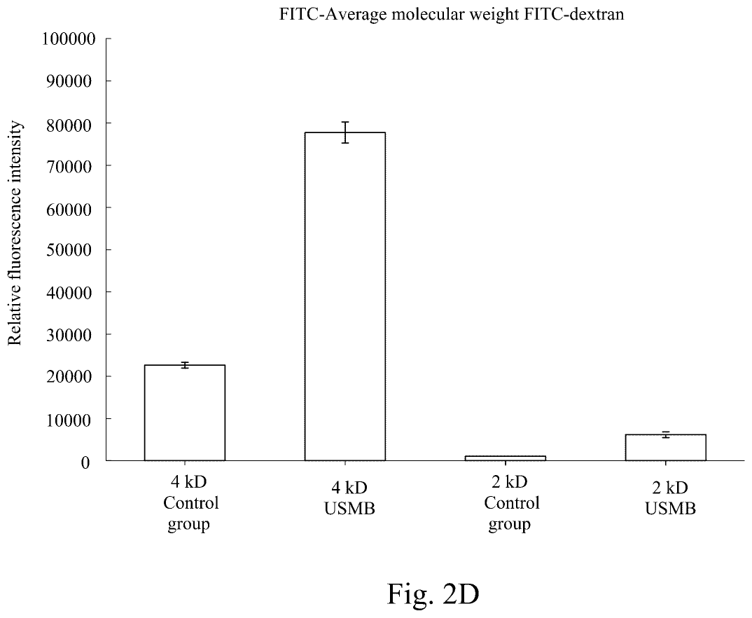

To understand the effect of ultrasound waves on bodily fluid collection through a round window membrane, a bodily fluid collection device was designed using material extrusion technology in 3D printing. As shown in FIGS. 2A, 2B, and 2C, the in vitro bodily fluid collection model 100 includes a connecting pipe 110, a dialysis membrane 120, and a fixing ring 130. The connecting pipe 110 is U-shaped, imitating the inner ear structure. The connecting pipe 110 includes a first end 111 and a second end 112 opposite to the first end 111. When the connecting pipe 110 is upright, a height of the first end 111 is higher than that of the second end 112. This is to imitate the pressure exerted on the round window membrane by the inner ear perilymph when the connecting pipe 110 is filled with liquid. The dialysis membrane 120 is disposed on the second end 112 of the connecting pipe 110, imitating the round window membrane. The molecular weight cutoff of the dialysis membrane 120 is 3.5 kDa. The molecular weight cutoff is chosen to be smaller than the molecular weight of the liquid to be tested, thus the molecular weight cutoff of the dialysis membrane 120 is not limited to the above-mentioned size. The fixing ring 130 includes a ring portion 131 and an accommodating portion 132. The ring portion 131 is sleeved on the outer wall of the second end 112 having the dialysis membrane 120 to fix the dialysis membrane 120. The accommodating portion 132 is disposed above the ring portion 131 to imitate the middle ear cavity and can accommodate microbubble composition MB or Schirmer strip SS. The microbubble composition MB was formed by mixing lipid microbubbles of sulfur hexafluoride with saline. The microbubble composition MB contained from 1.5×10⁷ microbubbles/mL to 5.6×10⁷ microbubbles/mL and had an average diameter of 2.5 μm. The above concentration range of microbubble composition MB was based on the manufacturer's information and obtained after a 10-fold dilution. Experiments had shown that the effect of administration within this range was similar.

During the experiment, firstly, a FITC-dextran solution with an average molecular weight of 4 kD or 20 kD was placed in the first end 111 of the connecting pipe 110 until a liquid level at the first end 111 was higher than a liquid level at the second end 112. Next, the experiment was divided into a USMB group (with microbubble composition MB placed in the accommodating portion 132) and a control group (without microbubble composition MB). The USMB group: a non-focused ultrasound (ST2000V, NepaGene, Japan) was in direct contact with the microbubble composition MB. Ultrasound was applied using an ultrasound probe USP with a diameter of 6 mm, a waveform of square wave, a duty cycle of 50%, an energy setting of 3 W/cm², and an irradiation duration of 60 seconds per irradiation for a total of 3 irradiations (as shown in FIG. 2B). Then, the accommodating portion 13 was washed three times with saline, and excess liquid was aspirated. Next, a portion of Schirmer strip SS in contact with the dialysis membrane 120 was placed flat on the dialysis membrane 120 for 30 minutes (as shown in FIG. 2C). Then, the Schirmer strip SS was cut into a plurality of small fragments and placed in PBS, then vortexed at 8° C. for 5 minutes. Next, these vortexed small fragments were centrifuged at 10000×g for 5 minutes to obtain a supernatant and a precipitate. Then, the precipitate (including cell debris and impurities) was removed, and the supernatant was analyzed for fluorescence.

The results are shown in FIG. 2D, the result was presented as relative fluorescence intensity after background subtraction. The relative fluorescence intensity of the 4 kD FITC-dextran in the control group was 22615, while the relative fluorescence intensity of the USBM group was 77655. This indicates that after the application of the ultrasound microbubble composition, the 4 kD FITC-dextran solution originally located in the connecting pipe 110 was significantly absorbed by the Schirmer strip SS. Furthermore, the relative fluorescence intensity of the 20 kD FITC-dextran in the control group was 882, while the relative fluorescence intensity of the USBM group was 5978. This shows that the control group could hardly penetrate the dialysis membrane 120, while the FITC-dextran solution could still be absorbed by the Schirmer strip SS after the application of the ultrasound microbubble composition. This indicates that simultaneous ultrasound application with the microbubble composition effectively induces the cavitation effect of the microbubble composition. The results also confirmed that it is feasible to absorb the imitated inner ear perilymph by applying ultrasound with the microbubble composition to the imitated round window membrane of the middle ear cavity.

### Example 2 Animal Experiment

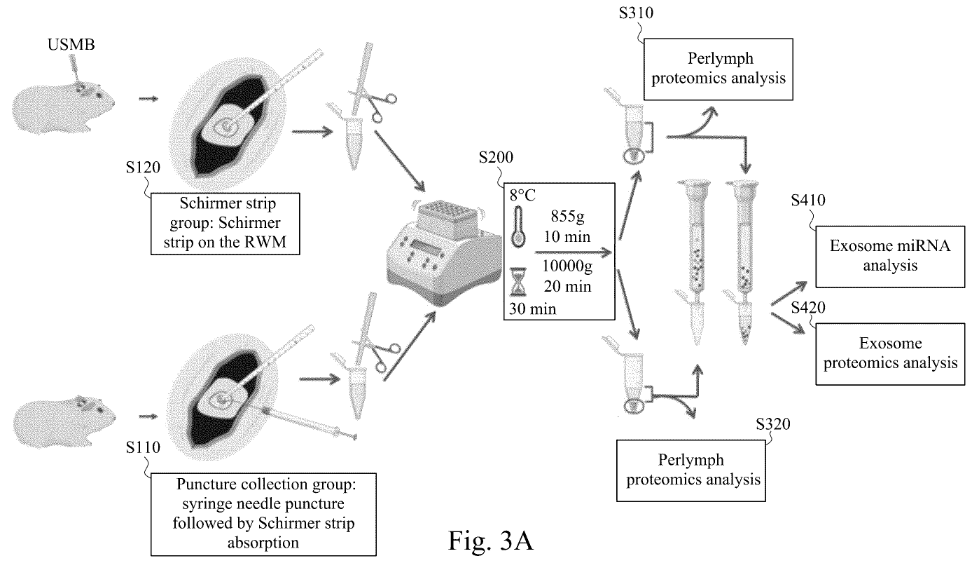

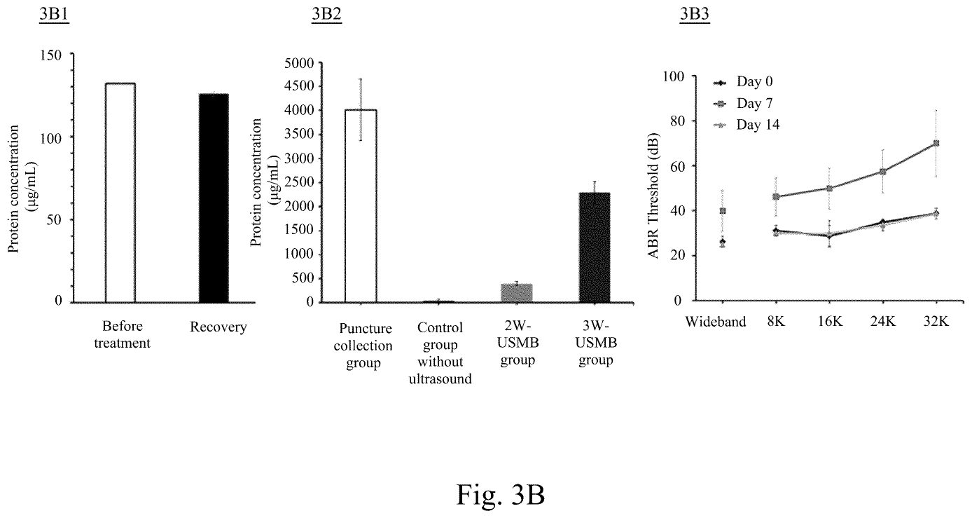

The present disclosure uses pigmented guinea pigs with an initial experimental weight of approximately 250 to 300 grams, having normal Preyer vocal reflexes. Please refer to FIG. 3A, the examples were divided into a puncture collection group (perilymph group) and multiple Schirmer strip groups (control group without ultrasound, 2W-ultrasound microbubble (USMB) group, and 3W-USMB group). The detailed procedure S110 for the puncture collection group was as follows: First, the guinea pigs were anesthetized. The entire surgical experiment was performed under a dissecting microscope while the guinea pigs were kept warm with a heating pad. Next, the skull was trepanned behind the guinea pig's ear to the middle ear cavity to expose the round window membrane (RWM). Next, the round window membrane was punctured from the middle ear cavity using a syringe needle, and one end of the Schirmer strip is placed on the side of the round window membrane facing the middle ear cavity for 30 minutes. Next, the Schirmer strip was cut into multiple small fragments and placed in PBS, and the small fragments were shaken at 8° C. for 30 minutes. Next, these shaken small fragments were centrifuged in two stages to obtain a supernatant for further analysis.

The detailed procedure S120 for multiple Schirmer strip groups is as follows: First, the guinea pigs were anesthetized, and the entire surgical experiment was performed under a dissecting microscope while kept warm with a heating pad. Next, the skull was trepanned behind the guinea pig's ear to the middle ear cavity to expose the round window membrane. Next, a microbubble composition containing 1.5×10⁷ microbubbles/mL to 5.6×10⁷ microbubbles/mL with an average diameter of 2.5 μm was formed by mixing sulfur hexafluoride lipid microbubbles with saline. The concentration range of the microbubble composition was obtained by diluting tenfold according to the manufacturer's specifications, and experiments had shown that the effect was equivalent within this range. Next, the microbubble composition was placed on the side of the guinea pig's round window membrane located in the middle ear cavity. Next, the guinea pigs were divided into a control group (without ultrasound, without microbubble composition placed), a 2W-USMB group, and a 3W-USMB group. In the ultrasound applying groups, a non-focused ultrasound (ST2000V, NepaGene, Japan) was used to directly contact the microbubble composition. Ultrasound was applied using the ultrasound probe with a diameter of 6 mm, a waveform of square wave, a duty cycle of 50%, an energy setting of 2 W/cm² (2W-USMB group) or 3 W/cm² (3W-USMB group), and an irradiation duration of 60 seconds per irradiation for a total of 3 irradiations. Next, the microbubble composition on the round window membrane was removed, washed three times with saline, and dried. Next, one end of a Schirmer strip was placed on the side of the round window membrane located at the middle ear cavity for 30 minutes. Next, the Schirmer strip was cut into multiple small fragments and placed in PBS, shaken at 8° C. for 30 minutes. Finally, these shaken small fragments were centrifuged in two stages to obtain a supernatant for further analysis.

Regarding the perilymph proteomic analysis: Stage 1, small fragments collected from a portion of the puncture collection group and from a portion of each of the multiple Schirmer strip groups were centrifuged at 855×g for 10 minutes to obtain a first supernatant and a first precipitate. Stage 2, the first supernatant was centrifuged at 10000×g for 20 minutes to obtain a second supernatant and a second precipitate (step S200). Then, the second precipitate (including cell debris) was removed, and the second supernatant was used for perilymph proteomic analysis (steps S310 and S320).

Regarding exosome proteomic analysis and miRNA analysis in perilymph: the small fragments obtained after shaking from the other portion of the puncture collection group and the small fragments obtained after shaking from the other portion of each of the multiple Schirmer strip groups were centrifuged at 855×g for 10 minutes to obtain a first supernatant and a first precipitate. Stage 2: The first supernatant was centrifuged at 10000×g for 20 minutes to obtain a second supernatant and a second precipitate (step S200). Next, the second precipitate (including cell debris) was removed, and the second supernatant was purified to obtain exosomes by a qEV column. The exosomes were then concentrated by centrifugation at 4000×g for 1 minute. Finally, exosome proteomic analysis and miRNA sequencing were performed (steps S410 and S420).

### Example 3

To establish a method for collecting perilymph with minimal damage to the round window membrane, a low-damage yet efficient substance for perilymph collection was selected. Paper was used as the substance for testing water absorption and residual protein. Schirmer strips were chosen, commonly used in dry eye patients to detect tear proteins and to absorb excess moisture in localized areas to keep them dry. A complete Schirmer strip was cut into a 2 mm wide piece, and PBS was aspirated until the strip was completely saturated before testing. To confirm the protein recovery rate of the Schirmer strip, a fixed volume of bovine serum albumin (BSA) of known concentration was aspirated onto the strip, and bicinchoninic acid protein assay (BCA protein assay) was used to confirm the protein concentration of the sample before and after treatment.

As shown in drawing 3B1 of FIG. 3, the results show that the protein concentration before treatment was 132 μg/mL, and the recovered concentration was 125 μg/mL, with a protein recovery rate of 95%, indicating that the use of Schirmer strip had a great protein recovery rate.

Furthermore, the present disclosure also tested substrates containing protein component that release proteins from the substrates. For example, even if a cotton swab or a paper point absorbs only a solution without protein component (such as PBS), protein component can still be detected after centrifugation, indicating that the protein component originated from the cotton swab or paper point itself. Experimental results (not shown) revealed that the release of protein component from cotton swabs and paper points severely affected the reliability of the experimental results. Therefore, substrates containing protein component and releasing their proteins are examples that the present disclosure aims to exclude.

### Example 4 Collecting Leaked Perilymph after Applying USMB on the Round Window Membrane

Following Example 2, in order to collect perilymph without damaging the round window membrane, skull was trepanned behind the guinea pig's ear to the middle ear cavity to expose the round window membrane. The groups were then divided into a puncture collection group (perilymph group) and multiple Schirmer strip groups (control group without ultrasound, 2W-USMB group, and 3W-USMB group). Each of samples was collected from each of the Schirmer strip groups for 30 minutes. After low-temperature extraction and centrifugation, protein concentration was determined using BCA protein analysis.

The results are shown in drawing 3B2 of FIG. 3B. Almost no protein was collected from the Schirmer strip in the control group without ultrasound. The 2W-USMB group yielded 400 μg/mL of protein, the 3W-USMB group yielded 2155 μg/mL, and the puncture collection group yielded 4000 μg/mL. This indicates that USMB was required for the collection of leaked perilymph through the Schirmer strip, and as the intensity of the USMB increased, the amount of protein that the Schirmer strip can collect also increased. Therefore, the subsequent example used 3W-USMB as the experimental condition.

### Example 5 the Impact of Perilymph Leakage on Hearing

To determine whether placing the Schirmer strip against the round window membrane would affect hearing, the wound was sutured after perilymph was collected. Hearing function was assessed in animals on days 0, 7, and 14 using auditory brainstem response (ABR), with wideband (click) and tone burst as test sources.

As shown in drawing 3B3 of FIG. 3B, hearing was slightly affected on day 7, possibly due to fluid retention in the inner ear caused by the wound. After the fluid was gradually absorbed by the tissue, hearing was almost the same as on day 0 after day 14. This indicates that using the Schirmer strip to collect perilymph after USMB injection did not cause irreversible damage to hearing.

### Example 6 Proteomic Analysis of Perilymph after Applying USMB

Based on the second supernatant obtained from the puncture collection group (perilymph group) and the 3W-USMB group in Example 2, perilymph proteomic analysis was performed. The protein concentration of the second supernatant from both the puncture collection group and the 3W-USMB group was first ensured by quantitative protein analysis, and then analyzed by LC-MS/MS, with the data from the two samples concatenated.

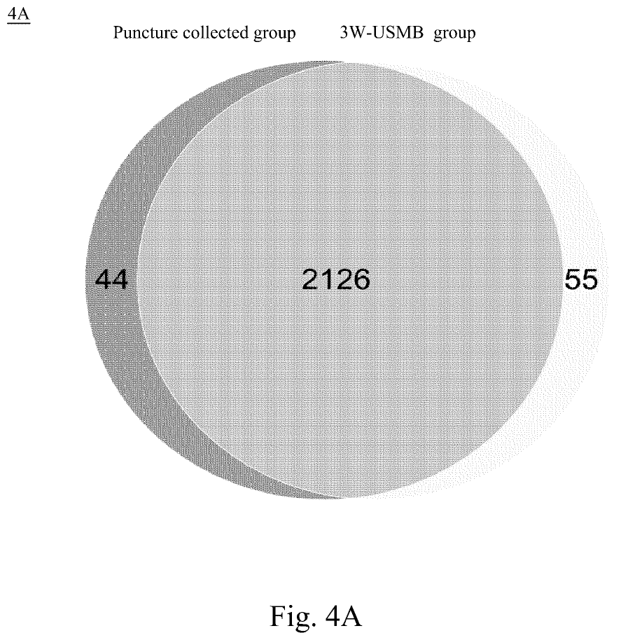

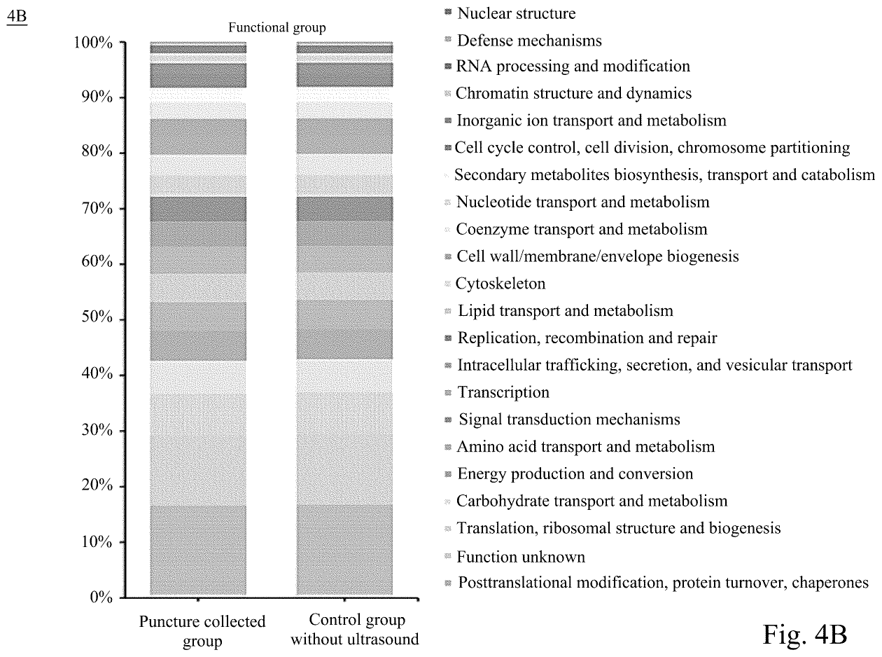

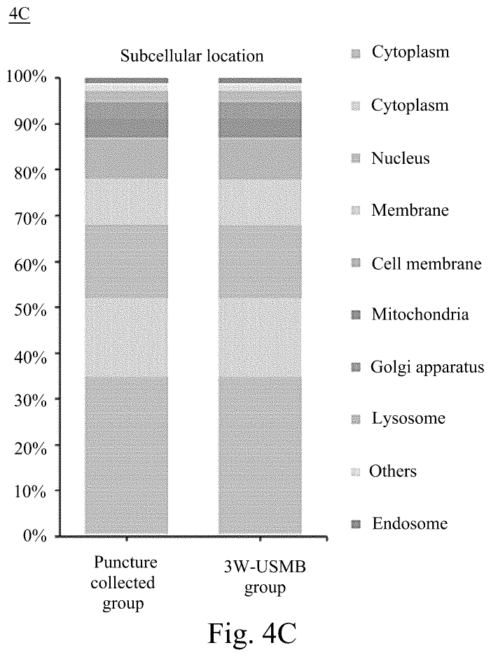

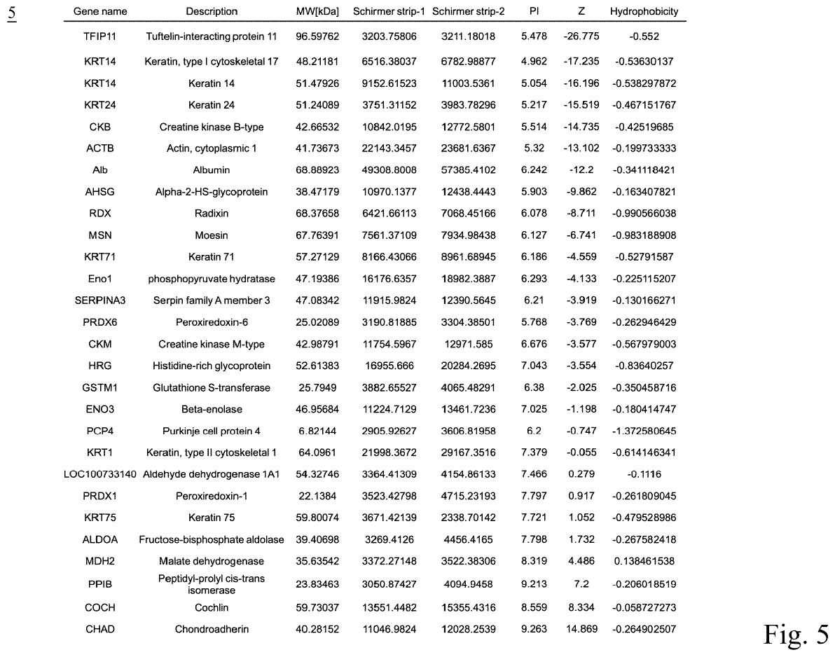

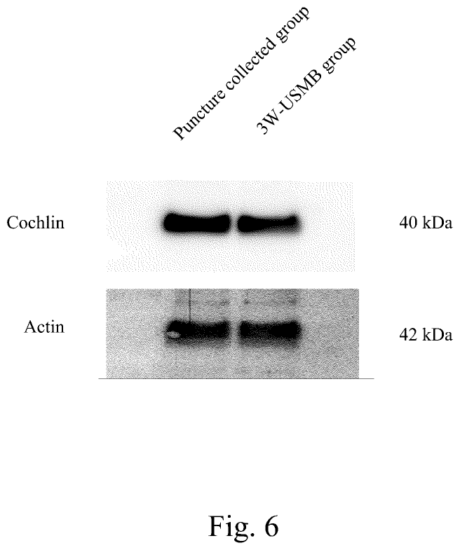

The results are shown in drawing 4A of FIG. 4A, drawing 4B of FIG. 4B, and drawing 4C of FIG. 4C. The puncture collected group contained 2170 proteins, and the 3W-USMB group contained 2181 proteins. The Venn diagram shows that 2126 proteins coexisted in both groups; 44 were found only in the puncture collected group, and 55 were found only in the 3W-USMB group. Furthermore, proteomic analysis reveals that among the top 25 of the 2126 proteins, cochlin protein (COCH) and albumin (drawing 5 of FIG. 5) were included. Albumin (Alb) was known to be the most abundant protein in the perilymph, and cochlin protein, in particular, was mainly found in the inner ear perilymph. In addition, apolipoprotein A1 (APOA1, molecular weight 29.3613 kDa) was also identified among the 2126 proteins. These results indicate that after applying ultrasound microbubbles (USMB) to a round window membrane, the perilymph collected via the Schirmer strip indeed reflected the true perilymph composition.

Next, Western blot analysis was used for further confirmation. The results as shown in drawing 6 of FIG. 6 indicate that cochlin protein was identifiable in both the puncture collection group and the 3W-USMB group. Therefore, cochlin protein can be sampled using the method disclosed herein. These results confirm that the ultrasound microbubble-assisted in vivo cochlear fluid liquid biopsy method was feasible for collecting and analyzing perilymph.

### Example 7 Characteristics of Perilymph-Derived Exosomes

The exosomes obtained from the puncture collection group (perilymph group) and 3W-USMB groups in Example 2 were purified by qEV column chromatography. The characterization and concentration of these exosomes were evaluated using scanning electron microscopy (SEM), dynamic light scattering (DLS), and Western blot.

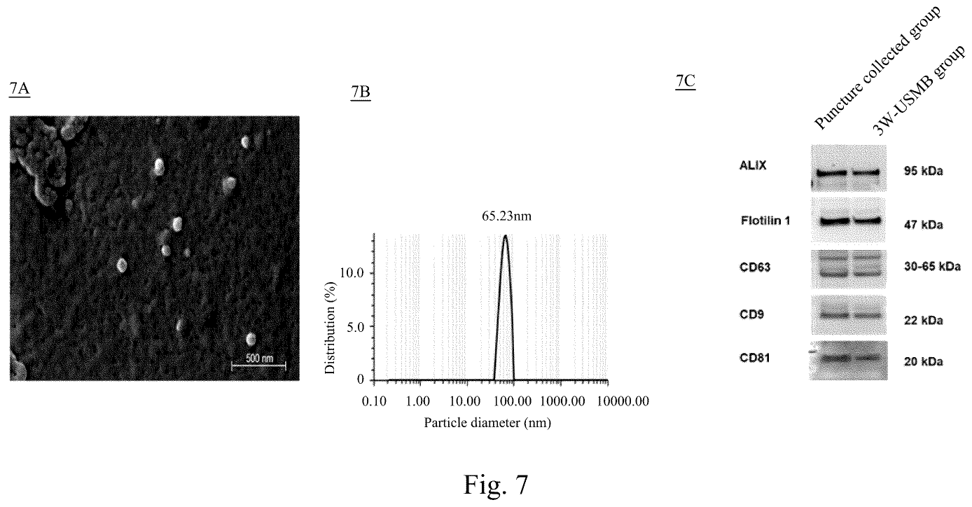

The results shows that perilymph-derived exosomes collected by Schirmer strip after USMB were visible using SEM (drawing 7A of FIG. 7). Subsequently, exosomes collected by Schirmer strip in the 3W-USMB group were detected by DLS at a size of approximately 65.23 nm (drawing 7B of FIG. 7) and a concentration of 1.19×10¹⁰/mL. Exosomes collected from the puncture collection group were approximately 55 nm in size (not shown) and at a concentration of 1.43×10¹¹/mL. Furthermore, Western blot analysis of exosome-related marker proteins revealed the expression of ALIX, Flotillin 1, CD63, CD9, and CD81 in the exosomes (drawing 7C of FIG. 7). This will help increase the likelihood of identifying potential biomarkers in the future. These results confirm that the method of ultrasound microbubble-assisted in vivo cochlear lymph liquid biopsy is effective for collecting exosomes, and the analysis is feasible.

### Example 8 Proteomic Analysis of Exosomes in Perilymph

Proteomic analysis was performed on the exosomes obtained from the puncture collection group and the 3W-USMB group in Example 2.

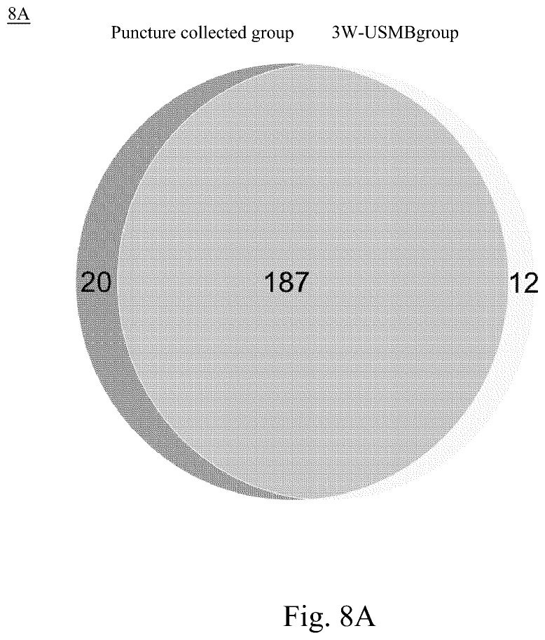

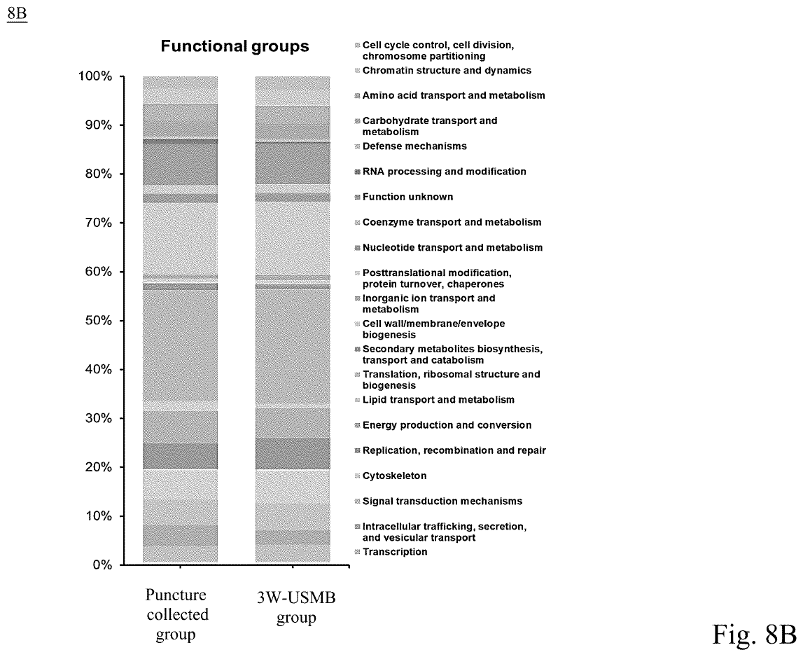

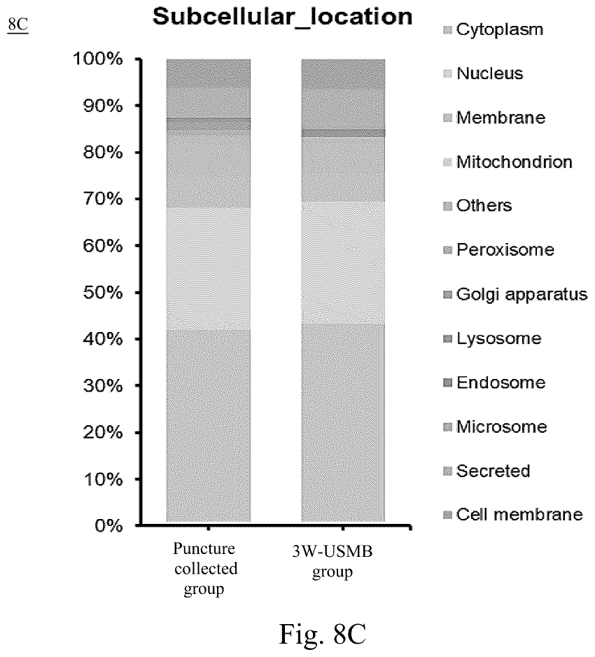

As shown in drawing 8A of FIG. 8A, a union of two datasets revealed that 187 exosome proteins were presented in both datasets. As shown in drawing 8B of FIG. 8B, the functional performance of exosome proteins was identical between the two groups (puncture collection group and 3W-USMB group), with minimal differences in percentage analysis. For example, among the 187 proteins, the percentages for “translation, ribosome structure and biogenesis” were 22.77% and 23.4%, respectively; for “posttranslational modification, protein turnover, and chaperones” were 14.66% and 15.06%, respectively; for “function unknown” were 8.38% and 8.33%, respectively; and for “energy production and conversion” were 6.54% and 6.09%, respectively.

Further analysis of the exosome protein distribution locations was conducted. Drawing 8C of FIG. 8C shows that the distribution locations were identical between the samples of the two groups, with little difference in percentage. The distribution locations were: cytoplasm (42.03% vs. 43.28%), nucleus (26.1% vs. 26.05%), membrane (6.78% vs. 6.3%), and mitochondrion (6.78% vs. 6.3%). This confirms that the method of ultrasound microbubble-assisted in vivo cochlear lymph liquid biopsy is feasible for collecting exosomes for protein biopsy analysis.

### Example 9 Mirna Analysis of Exosomes in Perilymph

miRNA analysis was performed on the exosomes obtained from the puncture collection group and the 3W-USMB group in Example 2.

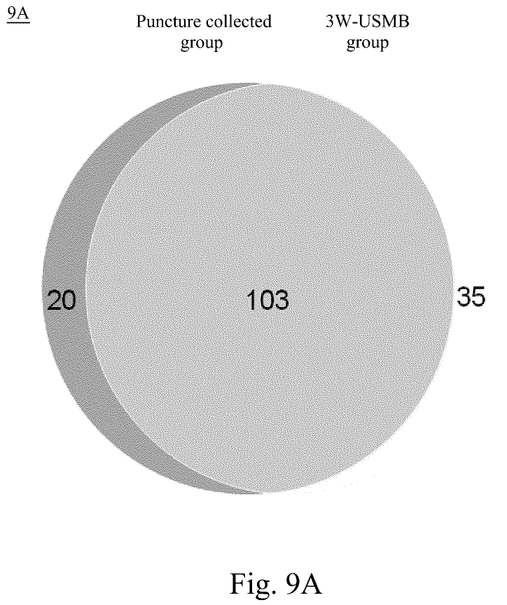

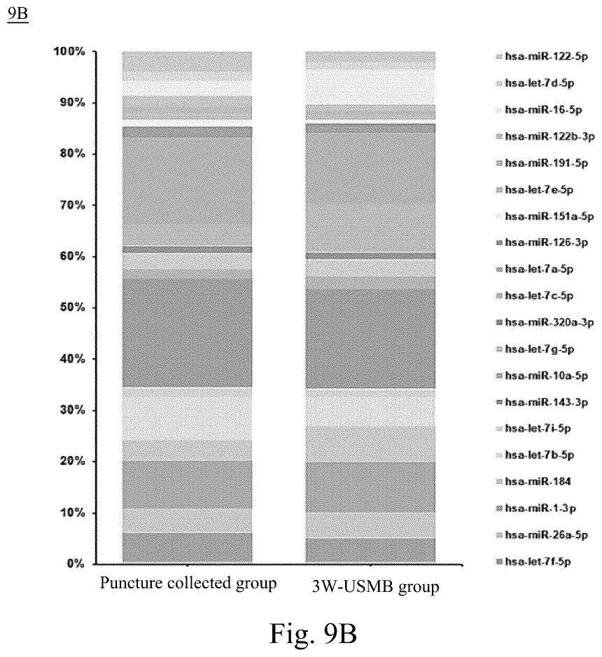

A union of two datasets revealed that 103 miRNAs were presented in both datasets, and analysis of the top 20 miRNAs by exosome expression was then conducted. The results are shown in drawing 9A of FIG. 9A and drawing 9B of FIG. 9B, the top 20 miRNAs exhibited similar expression levels. This confirms that the method of ultrasound microbubble-assisted in vivo cochlear lymph liquid biopsy is feasible for collecting exosomes for miRNA analysis.

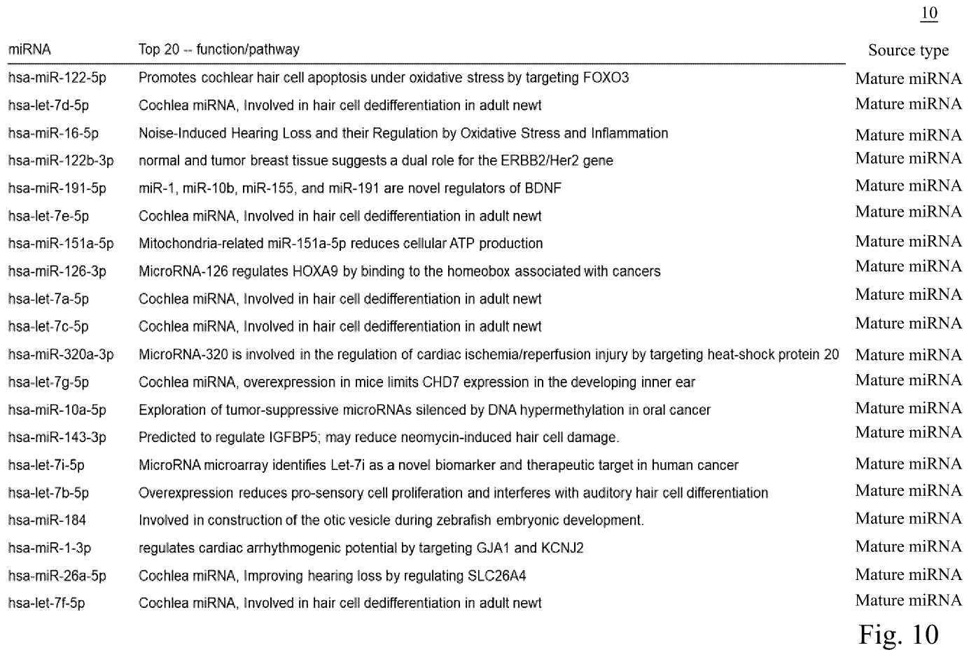

Furthermore, the top 20 miRNAs expression in exosomes all have inner ear-related gene regulatory functions. Specifically, TargetScan (http://www.targetscan.org/) was used to predict the genes that miRNAs in Schirmer strip samples might target, and PubMed (https://pubmed.ncbi.nlm. nih.gov) was used to search for experimental evidence supporting an interaction between the two to identify the potential roles of these miRNAs in the inner ear. The results, as shown in drawing 10 of FIG. 10, indicate that based on literature and target analysis, these miRNAs can be broadly categorized into four classes: miRNAs related to hair cell regeneration and survival, inner ear damage and protection, inner ear development and functional regulation, and inflammation and oxidative stress. Among them, miR-185-5p, miR-181a-5p, and miR-26a-5p had been confirmed to be highly expressed miRNAs in the cochlea.

miRNAs associated with hair cell regeneration and survival: let-7a-5p belongs to the let-7 family; miR-181a was involved in the regeneration of avian hair cells; insulin-like growth factor (IGFBP5) targeted by miR-143-3p can reduce the damage to hair cells caused by the ototoxic drug neomycin; miR-34a was associated with the loss of aging cochlear cells.

miRNAs associated with inner ear injury and protection: miR-16-5p, miR-185-5p, and miR-24-3p were reduced in the serum of patients with occupational noise-induced hearing loss (ONIHL); miR-205-5p, as a member of the miR-205 family, can serve as a biomarker for hearing loss caused by ototoxic injury.

miRNAs associated with inner ear development and functional regulation: brain-derived neurotrophic factor (BDNF) targeted by miR-191-5p can improve hearing in guinea pigs; fibroblast growth factor receptor 3 (FGFR3 ) targeted by miR-100-5p was associated with inner ear development.

miRNAs associated with inflammation and oxidative stress: miR210 was significantly downregulated in the middle ear effusion and serum of patients with effusion-type otitis media, and miR-210 regulated the inflammatory response by mediating HIF-1α. These miRNAs were not only identified as having an association with inner ear function through literature and target prediction, but were also successfully collected using Schirmer strips, demonstrating that the method of ultrasound microbubble-assisted in vivo cochlear lymph liquid biopsy has the potential to serve as a non-invasive sampling tool for biomarkers of inner ear diseases.

### Example 10 Applying Ultrasound Microbubble to Palpebra

The experiment was divided into a puncture collection group (perilymph group) and multiple Schirmer strip groups (control group without ultrasound, 2W-USMB group, and 3W-USMB group). The detailed procedures for the puncture collection group were as follows: First, the guinea pigs were anesthetized. The entire surgical experiment was performed under a dissecting microscope while the guinea pigs were kept warm with a heating pad. Next, lower palpebra of the guinea pig was pulled down and fixed with adhesive to the expose lower palpebra. Next, the experiment was divided into the puncture collection group and the multiple Schirmer strip groups. In the puncture collection group, the lower palpebra was punctured with a syringe needle, and one end of a Schirmer strip was placed on the lower palpebra for 30 minutes. In the multiple Schirmer strip groups, the concentration of microbubble composition ranged from 1.5×10⁷ microbubbles/mL to 5.6×10⁷ microbubbles/mL with an average diameter of 2.5 micrometers (same as Example 1). Next, the microbubble composition was placed on the lower palpebra. Next, groups were divided into three categories: a control group (without placing microbubble composition), a 2W-USMB group, and a 3W-USMB group. The groups receiving ultrasound that was non-focused ultrasound were in direct contact with the microbubble composition. A diameter of the ultrasound probe was 6 mm, the waveform was a square wave, the duty cycle was 50%, and the energy was set to either 2 W/cm² (2W-USMB group) or 3W/cm² (3W-USMB group). The irradiation duration was 60 seconds per irradiation, for a total of three irradiations. Next, the microbubble composition on the lower palpebra was removed, washed three times with saline solution, and dried.

Next, the Schirmer strips of the puncture collection group and multiple Schirmer strip groups were cut into multiple small fragments and placed into PBS, then vortexed at 8° C. for 30 minutes. Next, these vortexed small fragments were centrifuged in two stages to obtain supernatants for further analysis. The procedures for exosome proteomics analysis and miRNA analysis are the same as in Example 2 and will not be repeated herein.

### Example 11 Applying Ultrasound Microbubble to Skin Surface

The experiment was divided into a puncture collection group (perilymph group) and multiple Schirmer strip groups (control group without ultrasound, 2W-USMB group, and 3W-USMB group). The detailed procedures for the puncture collection group were as follows: First, the guinea pigs were anesthetized. The entire surgical experiment was performed under a dissecting microscope while the guinea pigs were kept warm with a heating pad. Next, the fur on the thigh of the guinea pig was shaved to expose the thigh skin. Next, the experiment was divided into the puncture collection group and the multiple Schirmer strip groups. In the puncture collection group, the thigh skin was punctured with a syringe needle, and one end of the Schirmer strip was placed on the lower palpebra for 30 minutes. In the multiple Schirmer strip groups, the concentration of microbubble composition ranged from 1.5×10⁷ microbubbles/mL to 5.6×10⁷ microbubbles/mL with an average diameter of 2.5 micrometers (same as Example 1). Next, the microbubble composition was placed on the thigh skin. Next, groups were divided into three categories: a control group (without placing microbubble composition), a 2W-USMB group, and a 3W-USMB group. The groups receiving ultrasound that was non-focused ultrasound were in direct contact with the microbubble composition. A diameter of the ultrasound probe was 6 mm, the waveform was a square wave, the duty cycle was 50%, and the energy was set to either 2 W/cm² (2W-USMB group) or 3W/cm² (3W-USMB group). The irradiation duration was 60 seconds per irradiation, for a total of three irradiations. Next, the microbubble composition on the thigh skin was removed, washed three times with saline solution, and dried.

Next, the Schirmer strips were cut into multiple small fragments and placed into PBS, then vortexed at 8° C. for 30 minutes. Next, these fragments were centrifuged in two stages to obtain supernatants for further analysis. The procedures for exosome proteomics analysis and miRNA analysis are the same as in Example 2 and will not be repeated herein.

The present disclosure provides a method of microbubble-assisted liquid biopsy, which collects and analyzes bodily fluids leaking from the body surface by an ultrasonic microbubble. Proteomic analysis of samples from cochlear perilymph liquid biopsy revealed an abundance of various known inner ear perilymph-related proteins, confirming that the samples were originated from perilymph. Furthermore, purification of exosomes from the samples revealed that perilymph-derived exosomes can be collected using 3W-USMB, and miRNA sequencing showed the presence of numerous known miRNAs associated with inner ear function, demonstrating its enormous potential in clinical diagnosis and biomarker discovery.

While the disclosure has been described by way of example(s) and in terms of the preferred embodiment(s), it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A method of ultrasound microbubble-assisted liquid biopsy, comprising:
providing an individual to be tested;
providing a microbubble composition, wherein the microbubble composition comprises a medium and a plurality of microbubbles dispersed in the medium;
placing the microbubble composition on a surface of the individual to be tested;
placing an ultrasonic device in contact with the microbubble composition, and applying non-focused ultrasound to the microbubble composition to raise a temperature of the surface by no more than 3° C.; and
placing an absorbent substrate on the surface of the individual to be tested and absorbing a liquid located inside the individual to be tested, wherein the absorbent substrate is free of protein component.

2. The method of claim 1, further comprising:
placing the absorbent substrate in a solvent and centrifuging to obtain a supernatant and a precipitate, the precipitate comprising cell debris; and
performing a substance analysis on the supernatant.

3. The method of claim 1, further comprising:
placing the absorbent substrate in a solvent and centrifuging to obtain a supernatant and a precipitate, the precipitate comprising cell debris;
separating the supernatant by an exosome filtering to obtain a plurality of exosomes; and
analyzing proteins, nucleotides, or a combination thereof of each one of the plurality of exosomes.

4. The method of claim 3, wherein the exosome filtering comprises size exclusion chromatography, ultracentrifugation, sucrose density gradient centrifugation, ultrafiltration, polymer precipitation, immuno-magnetic bead capture, or microfluidic chip method.

5. The method of claim 1, wherein the medium comprises water, gel, or a combination thereof.

6. The method of claim 1, wherein a material of the plurality of microbubbles comprises albumin, polymer, lipid or a combination thereof.

7. The method of claim 1, wherein particle sizes of the plurality of microbubbles are from 0.5 μm to 2.5 μm.

8. The method of claim 1, wherein the ultrasonic device generates from 1 W/cm² to 5 W/cm².

9. The method of claim 1, wherein concentrations of the plurality of microbubbles in the microbubble composition range from 1×10⁶ particles/mL to 2×10⁸ particles/mL.

10. The method of claim 1, wherein the surface of the individual to be tested comprises skin, palpebra, nasal mucosa, or oral mucosa.

11. The method of claim 1, wherein the absorbent substrate comprises Schirmer strip, low-lint lab wipes, cellulose filter paper, glass fiber filter paper, cleanroom wipes, or a combination thereof.

12. A method of ultrasound microbubble-assisted liquid biopsy, comprising:
providing a microbubble composition, wherein the microbubble composition comprises a medium and a plurality of microbubbles dispersed in the medium;
providing a model, comprising:
a connecting pipe configured to imitate an inner ear structure, the connecting pipe comprising a first end and a second end opposite to the first end, configured to imitate inner ear structure;
a dialysis membrane disposed on the second end of the connecting pipe, configured to imitate round window membrane; and
a fixing ring comprising a ring portion and an accommodating portion, wherein the ring portion sleeved on an outer wall of the second end having the dialysis membrane, configured to imitate middle ear cavity;
filling a liquid into the connecting pipe;
placing the microbubble composition in the accommodating portion and located on the dialysis membrane;
placing an ultrasonic device in contact with the microbubble composition, and applying a non-focused ultrasound to the microbubble composition to raise a temperature of the dialysis membrane by no more than 3° C.; and
placing an absorbent substrate on the dialysis membrane, and absorbing the liquid in the connecting pipe, wherein the absorbent substrate is free of protein component.

13. A method of ultrasound microbubble-assisted liquid biopsy, comprising:
providing an individual to be tested, and then trepanning from a skull behind an ear to the middle ear cavity to expose a round window membrane;
providing a microbubble composition, wherein the microbubble composition comprises a medium and a plurality of microbubbles dispersed in the medium;
placing the microbubble composition on a surface of the round window membrane of the individual to be tested, and the surface located in the middle ear cavity;
placing an ultrasonic device in contact with the microbubble composition, and applying non-focused ultrasound to the microbubble composition to raise a temperature of the surface by no more than 3° C.; and
placing an absorbent substrate on the surface of the round window membrane of the individual to be tested, the surface located in a middle ear cavity, and absorbing a lymph located in an inner ear of the individual to be tested, wherein the absorbent substrate is free of protein component.

14. The method of claim 13, further comprising:
placing the absorbent substrate in a solvent and centrifuging to obtain a supernatant and a precipitate, the precipitate comprising cell debris; and
performing a substance analysis on the supernatant.

15. The method of claim 13, further comprising:
placing the absorbent substrate in a solvent and centrifuging to obtain a supernatant and a precipitate, the precipitate comprising cell debris;
separating the supernatant by an exosome filtering to obtain a plurality of exosomes; and
analyzing proteins, nucleotides, or a combination thereof of each one of the plurality of exosomes.

16. The method of claim 15, wherein the exosome filtering comprises size exclusion chromatography, ultracentrifugation, sucrose density gradient centrifugation, ultrafiltration, polymer precipitation, immuno-magnetic bead capture, or microfluidic chip method.

17. The method of claim 13, wherein the medium comprises water, gel or a combination thereof.

18. The method of claim 13, wherein a material of the plurality of microbubbles comprises albumin, polymer, lipid or a combination thereof.

19. The method of claim 13, wherein particle sizes of the plurality of microbubbles are from 0.5 μm to 2.5 μm.

20. The method of claim 13, wherein concentrations of the plurality of microbubbles in the microbubble composition range from 1×10⁶ particles/mL to 2×10⁸ particles/mL.
